(19) United States
(12) Patent Application Publication
(10) Pub. No.: US 2011/0229879 A1
(43) Pub. Date: Sep. 22, 2011

(54) METHODS AND COMPOSITIONS FOR NUCLEAR STAINING

(75) Inventor: Charles J. CHURUKIAN, Rochester, NY (US)

(73) Assignee: UNIVERSITY OF ROCHESTER, Rochester, NY (US)

(21) Appl. No.: 13/052,791

(22) Filed: Mar. 21, 2011

Related U.S. Application Data
(60) Provisional application No. 61/315,483, filed on Mar. 19, 2010.

(51) Int. Cl. C12Q 1/68

(52) U.S. Cl. 435/6.1

(57) ABSTRACT

The present invention relates to compositions, methods, and kits suitable for detecting nucleic acids in a biological sample. The nuclear staining composition of the present invention contains a pH buffering reagent, a solubilizing reagent, a basic dye, and an aqueous medium. The composition can be used alone to detect nucleic acids in a biological sample or in combination with other histological dyes for nuclear counterstaining.

This application claims the benefit of U.S. Provisional Patent Application Ser. No. 61/315,483, filed Mar. 19, 2010, which is hereby incorporated by reference in its entirety.

### FIELD OF THE INVENTION

The present invention relates to compositions and methods suitable for detecting nuclear elements in a biological sample.

### BACKGROUND OF THE INVENTION

Histochemical procedures performed on surgical, autopsy, and biopsy tissue and cell samples for diagnostic and research purposes generally involve the use of a nuclear counterstain to delineate tissue architecture and cellular detail. The nuclear counterstain is usually a dark color to contrast a lighter dye used to label the cytoplasmic or extracellular structures of interest.

A nuclear counterstain is the desired stain for a number of histological staining procedures including, for example, Gomori's reticulum, Pearl's ferric iron, Alcian blue for acidic mucins, Jones basement membrane, Churukian's ammonical silver for melanin, melanin bleach, and Lillie's ferrous iron uptake method for melanin. While nuclear fast red is most commonly used in methods requiring a red nuclear counterstain, this dye involves incubations periods of 5-10 minutes, and often fades within just weeks of staining. In addition, nuclear fast red is not compatible with all of the above noted staining procedures and has limited stability in solution. Accordingly, there is a need in the art for a nuclear dye that requires shorter incubation times, has increased compatibility with other staining procedures, is resistant to fading, and has long term stability. These needs are particularly, though certainly not exclusively, applicable for red nuclear dyes.

The present invention is directed to overcoming these and other deficiencies in the art.

### SUMMARY OF THE INVENTION

A first aspect of the present invention relates to a composition for detecting nucleic acids in a biological sample. This composition contains a pH buffering reagent that maintains the composition at a pH of less than 5. The composition further contains a solubilizing reagent, a basic dye, and an aqueous medium.

A second aspect of the present invention relates to a method of detecting nucleic acids in a biological sample that involves exposing the biological sample to a composition of the present invention under conditions effective to label the nucleic acids in the biological sample. The method further involves viewing the biological sample using light microscopy to detect the nucleic acids in the biological sample.

A third aspect of the present invention relates to a kit containing the composition of present invention and instructions for using the composition for detecting nucleic acids in a biological sample.

As described herein, the composition of the present invention offers many advantages over currently available nuclear dye solutions such as nuclear fast red. Firstly, the composition of the present invention does not precipitate in solution and has a shelf-life of at least one year. In addition, nuclear staining with the composition of the present invention achieves brighter, more brilliant nuclear staining showing superior tissue architecture and cellular detail within seconds of exposure. Finally, unlike conventional dyes that can weaken within weeks of staining, the nuclear staining composition of the present invention is extremely lightfast, with no significant fading observed over time.

### BRIEF DESCRIPTION OF THE DRAWINGS

FIGS. 1A-1B are light microscopy images of reticulum fiber staining in liver tissue sections using Gomori's reticulum staining procedure. Following staining for the reticulum, nuclear counterstaining was carried out using nuclear fast red solution (FIG. 1A) for five minutes and the strong fast red solution (0.1% pararosaniline solution) of the present invention (FIG. 1B) for 10 seconds.

FIGS. 2A-2B are light microscopy images of liver sections stained for iron using the Perl's ferric iron method. Following the staining procedure for the detection of iron, nuclear counterstaining was carried out using nuclear fast red solution (FIG. 2A) for 5 minutes and the strong fast red solution of the present invention (FIG. 2B) for 10 seconds.

FIGS. 3A-3B are light microscopy images of small bowel sections stained with alcian blue for acidic mucins. Following staining with alcian blue, nuclear counterstaining was carried out using nuclear fast red solution (FIG. 3A) for 5 minutes and the strong fast red solution of the present invention (FIG. 3B) for 20 seconds.

### DETAILED DESCRIPTION OF THE INVENTION

A first aspect of the present invention relates to a composition for detecting nucleic acids in a biological sample. The composition contains a pH buffering reagent that maintains the composition at a pH of less than 5. The composition further contains a solubilizing reagent, a basic dye, and an aqueous medium.

In accordance with this aspect of the present invention, the composition contains a pH buffering reagent that maintains the composition at a pH of less than 5. More preferably, the pH buffering reagent maintains the composition at a pH of less than 3. Even more preferably, the pH buffering reagent maintains the composition at a pH of between 2.3-2.7.

Any weak acid or weakly acidic buffer can be used as a pH buffering reagent in the composition of the present invention. Suitable pH buffering reagents include, but are in no way limited to lactic acid, acetic acid, citrate acid, oxalic acid, formic acid, hydrochloric acid, acetate buffer, citric acid/disodium phosphate buffer, sulfuric acid/sodium phosphate buffer, malonic acid/sodium hydroxide buffer, sodium acetate/acetic acid buffer, acid phosphate/hydrochloric acid buffer, and hydrochloric acid/sodium citrate buffer. The pH of the buffering reagent is preferably about 2.5, although buffers having a pH of about 2.3 to about 2.7 are also suitable for use. In one embodiment of the present invention, the composition contains lactic acid as the pH buffering reagent at a concentration of about 0.5% to about 0.7% by volume. Ideally, the lactic acid concentration of the composition of the present invention is at about 0.6% by volume. In an alternative embodiment of the present invention, the composition contains acetic acid as the pH buffering reagent at a concentration of about 1% to about 5% by volume. Ideally, the acetic acid concentration of the composition of the present invention is about 3% by volume.

The composition of the present invention also contains a solubilizing reagent. Suitable solubilizing reagents include low molecular weight emulsifiers or surfactants, including PEG-ylated sorbitan fatty acid esters, such as polysorbate 20 (Tween 20®). Alternatively, the solubilizing reagent can be a non-ionic surfactant having a hydrophilic polyethylene oxide group such as TritonX-100®. The concentration of the solubilizing reagent in the composition of the present invention is about 0.008% to about 0.05%. More preferably, the concentration of the solubilizing reagent in the composition of the present invention is about 0.01% to about 0.04%. In one embodiment of the present invention, the composition contains polysorbate 20 at a concentration of about 0.01% to about 0.04% by volume. More ideally, the polysorbate 20 concentration of the composition of the present invention is about 0.025% by volume. In an alternative embodiment of the present invention, the composition contains TritonX-100® at a concentration of about 0.01% to about 0.04% by volume. More ideally, the TritonX-100® concentration of the composition of the present invention is about 0.025% by volume.

The composition of the present invention further contains a basic dye. As used herein, a basic dye is any dye which is positively charged and binds to negatively charged tissue components. Basic dyes have amino groups or alkylamino groups as their auxochromes, giving them an overall positive charge. Numerous basic dyes for histological applications are known and readily available in the art. A suitable basic dye for use in the composition of the present invention may be chosen based on its color and the desired color for detecting nucleic acid components within a biological sample. Suitable basic red dyes that can be used in the composition of the present invention to achieve a red nuclear stain include, without limitation pararosanilin, rosaniline, fuchsine, new fuchsine (also known as magenta III), magenta II, safranin O, neutral red, rhodamine B, pyronin B, pyronin G, and iodonitrotetrazolium chloride. Suitable basic blue dyes for use in the composition of the present invention to achieve a blue nuclear stain include, without limitation, crystal violet, Victoria blue 4R, Victoria blue B, Victoria blue R, methylene blue, Nile blue A, toluidine blue O, azure A, azure B, azure C, nitro blue tetrazolium, night blue, alcian blue, gallamin blue, and gallocyanin. Suitable basic blue/violet or violet dyes for use in the composition of the present invention to achieve a blue/violet or violet nuclear stain include, without limitation, thionin, Hoffman's violet, methyl violet 2B, methyl violet 6B, mauveine, and ethyl violet. Suitable basic green dyes for use in the composition of the present invention to achieve a green nuclear stain include, without limitation, malachite green, methylene green, methyl green, and iodine green. Suitable basic yellow dyes for use in the composition of the present invention to achieve a yellow nuclear stain include, without limitation, Thioflavine T, alcian yellow, and Auramine O. A suitable basic brown dye for use in the composition of the present invention to achieve a brown nuclear stain includes, without limitation, bismark brown Y. These and other basic dyes that are known in the art and are commercially available (e.g., Sigma-Aldrich, Fischer Scientific), are suitable for use in the present invention.

The concentration of the basic dye in the composition of the present invention is between about 0.02% and about 0.2% by weight. The dye can also be present in an amount of about 0.04% to about 0.18% by weight or 0.05% to 0.15% by weight.

In one embodiment of the present invention, the composition contains pararosanilin at a concentration of about 0.05% to about 0.15% by weight. More ideally, the concentration of pararosanilin in the composition of the present invention is 0.1% by weight.

The composition of the present invention further contains an aqueous medium, such as, for example, deionized water.

The composition of the present invention has a pH of about 2 to about 3. More preferably, the pH of the composition is between about 2.4 to about 2.6. Most preferably, the pH of the composition of the present invention is about 2.5.

A significant benefit of the composition of the present invention is its stability over time. Unlike other commonly used dyes, such as nuclear fast red which has a limited shelf-life of about four months because of precipitation, the composition of the present invention is stable for at least one year at room temperature. In addition to its enhanced stability, the composition of the present invention, when used for staining the nuclear elements, has a much higher degree of lightfastness. Whereas the nuclear fast red stain begins to weaken considerably within weeks of staining, no weakening in the intensity of stain achieved with the composition of the present invention has been observed.

As described herein, the composition of the present invention is suitable for the detection of nucleic acids in a biological sample. The composition may be used alone (i.e., primary stain) or as a counterstain (i.e., secondary stain) to other histological stains.

A second aspect of the present invention relates to a method of detecting nucleic acids in a biological sample that involves exposing the biological sample to a composition of the present invention under conditions effective to label the nuclear acids in the biological sample. The method further involves viewing the biological sample using light microscopy to detect the nucleic acids in the biological sample.

In accordance with this aspect of the present invention, the biological sample can be any histological plant or animal tissue or cell sample. For example, the biological sample may be an autopsy, surgical, or biopsy sample from a human or animal subject taken for diagnostic or research purposes. Methods of preparing tissue and cell samples for histological analysis are well known in the art and will vary depending upon the primary histological staining procedures to be applied. The biological sample may be fresh, frozen, or fixed. Typically, however, the biological sample is a fixed biological sample. Commonly used fixatives suitable for histological analysis include 10% buffered neutral formalin, 10% formalin-sodium acetate, 10% formalin-alcohol, 10% formalin-calcium, modified Zenker's solution, Bouin's solution, Carnoy's solution, Lillie's B-5 fixative, and zinc formalin. Following fixation, the fixative is removed through a series of washing steps using deionized water or a neutral buffered solution. If the tissue is embedded in paraffin or plastic, excess water is first removed by dehydration via sequential washes or incubations in a gradient of ethanol solutions. After dehydration, the tissue sample may be embedded in paraffin, nitrocellulose or a variety of other formulations of plastic, and sectioned to the desired thickness using a standard microtome, ultratome, or cryostat. The tissue sections are then mounted to microscope slides for further analysis.

Once the biological sample has been fixed, embedded, and sectioned, it can be prepped for histological staining and analysis (e.g., deparaffinized and rehydrated). Once prepped, the biological sample on the slide is exposed to the composition of the present invention for staining and detecting of the nucleic acids within the sample. In accordance with the method of the present invention, a prepared tissue or cell sample is exposed to the composition of the present invention for a period of about 1 to about 60 seconds. Preferably, the exposure time is less than 60 seconds. More preferably, the exposure time is between about 5 and about 20 seconds.

Following exposure of the tissue or cell biological sample to the composition of the present invention, the sample may be washed one or more times to remove any excess staining solution from the sample. These washes can be carried out in water or a neutral buffered solution. Finally, the biological sample may be dehydrated and coverslipped for viewing using a light microscope.

The composition of the present invention may be used alone to detect or label nucleic acids in a biological sample as described above. In an alternative embodiment of the present invention, the composition is used to counterstain a biological sample stained with one or more other dye solutions capable of differentially labeling non-nucleic acid cellular components of the biological sample. In accordance with this embodiment of the invention, the prepared biological tissue or cell sample is first incubated with the one or more solutions capable of differentially labeling a non-nucleic acid cellular component, such as cellular pigments, metals, proteins, organelles, carbohydrates, mucopolysaccharides, connective tissue, and lipids. After the one or more non-nucleic acid cellular components have been labeled, the biological sample is then exposed to the composition of the present invention to counterstain the nucleic acid elements of the biological sample. Additional histochemical staining can be carried out after the nuclear counterstain if necessary. Once both non-nucleic acid and nucleic acid elements of the sample have been differentially labeled, both can be viewed simultaneously using light microscopy.

In accordance with this aspect of the invention, the basic dye of the composition should be selected to contrast with the dye solution used to stain the non-nucleic acid elements of the sample. For example, when the non-nucleic acid component or components of the biological sample are labeled with alcian blue solution, methenamine-silver nitrate solution, ammonical silver solution, hydrochloric acid-potassium ferrocyanide solution, or potassium ferricyanide/acetic acid solution, a red nuclear counter stain is desirable. Accordingly, a basic red dye, including any of those described supra, should be selected for the composition of the present invention. Likewise, when the non-nucleic acid component or components of the biological sample are stained red (e.g., trichrome stain, Van Gieson's solution, rhodanine copper solution, etc.), a non-red nuclear counterstain is desirable. In this embodiment, any of the non-red basic dyes described supra can be selected for incorporation into the composition of the present invention.

Biological samples that have been exposed to the composition of the present invention can be viewed using basic light microscopy techniques well known in the art. Typically a bright-field light microscope is used. An oil immersion lens may be employed if desired to enhance viewing at high magnifications.

Another aspect of the present invention relates to a kit containing the composition of present invention and instructions for using the composition for detecting nucleic acids in a biological sample. The kit may also contain one or more histological staining solutions, or the components necessary for making one or more histological staining solutions for labeling non-nucleic acid cellular elements in the biological sample, and instructions for performing both the labeling of non-nucleic acid and nucleic acid elements of a biological sample. In one embodiment of the present invention, the kit may contain one or more histological staining solutions selected from the group consisting of an Alcian blue solution, methenamine-silver nitrate solution, ammonical silver solution, hydrochloric acid-potassium ferrocyanide solution, potassium ferricyanide/acetic acid solution, and the composition of the present invention containing a basic red dye.

The kit may also include one or more of the following: suitable fixative solution, buffered wash solutions, embedding materials, ethanol, and blocking reagents.

### EXAMPLES

### Example 1

### Preparation of 0.1% Pararosanilin Solution

The 0.1% pararosanilin solution of the present invention is prepared by combining 0.1 gram pararosanilin (C.I. 52000) with 100 mL 0.6% lactic acid solution and 0.1 mL of 25% polysorbate 20. The pH of this solution is approximately 2.50.

### Comparative Example 2

### Preparation of Nuclear Fast Red Solution

Nuclear fast red solution was prepared by dissolving 0.1 gm nuclear fast red in 100 mL of 5% solution of aluminum sulfate with the aid of heat. The solution was cooled, filtered, and a few grains of thymol were added as a preservative.

### Example 3

### Modified Gomori's Method for Reticulin Staining with Nuclear Counterstain in Liver Tissue Sections

Liver tissue samples were fixed in 10% buffered neutral formalin and embedded in paraffin. Paraffin sections were cut at 5 μm. For reticulum staining, sections were deparaffinized, hydrated, and then oxidized using acidified potassium permanganate (0.3 gm potassium permanganate, 100 mL distilled water, and 0.2 mL sulfuric acid) for three minutes. Sections were rinsed in distilled water and reduced with 1% potassium metabisulfite for 1 minute. Sections were rinsed with running tap water for three minutes and then rinsed with four changes of distilled water prior to incubating with Ammoniacal silver solution for two minutes. Tissue sections were then rinsed three times and reduced in 10% formalin for one minute. Following the incubation in formalin, the sections were washed with running tap water for one minute and then rinsed with two changes of distilled water. Next, sections were toned in 0.2% gold chloride for 30 seconds, rinsed with distilled water, and fixed in 2% sodium thiosulfate for one minute. Sections were then washed with tap water and two changes of distilled water.

Following reticulin staining, tissue sections were counterstained with 0.1% nuclear fast red solution of Comparative Example 2 for 5 minutes or the 0.1% pararosaniline solution of Example 1 for 10 seconds. The sections were rinsed three times with distilled water, dehydrated, cleared with xylene and mounted with synthetic resin for viewing.

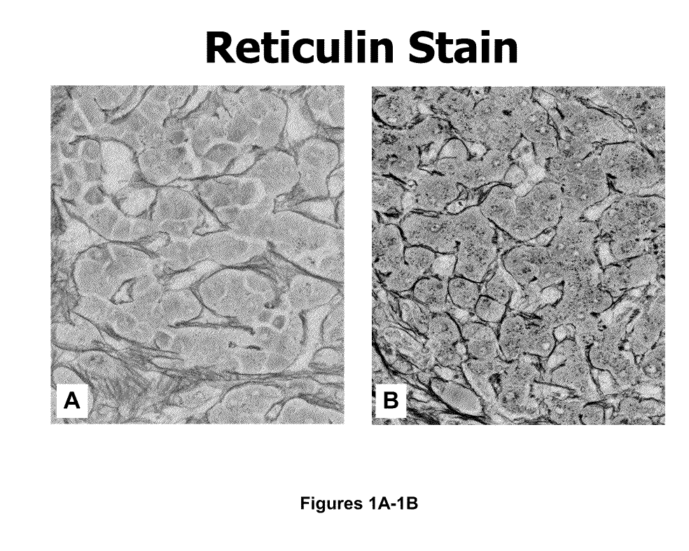

FIGS. 1A and 1B show a comparison of the reticulin staining with nuclear fast red counterstain (FIG. 1A) and 0.1% pararosaniline nuclear counterstain (“strong fast red”) (FIG. 1B). The nuclear staining achieved with the 0.1% pararosaniline solution is much brighter than that achieved with the nuclear fast red, showing superior tissue architecture. A sharper and clearer contrast between the primary stain (reticulin staining) and secondary stain (nuclear staining) is observed with the pararosaniline nuclear stain of Example 1 compared to the nuclear fast red stain of Comparative Example 2. In addition, no masking of the primary stain or non-specific staining was observed with the pararosaniline nuclear stain.

### Example 4

### Perl's Method for Ferric Iron Staining with Nuclear Counterstain in Liver Tissue Sections

Liver tissue was fixed in 10% buffered neutral formalin, embedded in paraffin, and cut at 5 μm for staining. Slides containing the liver tissue sections were deparaffinized and hydrated in distilled water. Slides were then placed in hydrochloric acid-potassium ferrocyanide solution (20 mL 2% hydrochloric acid and 20 mL 1% potassium ferrocyanide) for 30 minutes at room temperature. Following this incubation, the slides were rinsed in five changes of distilled water and counterstained with 0.1% nuclear fast red solution of Comparative Example 2 for 5 minutes or the 0.1% pararosaniline solution of Example 1 for 10 seconds. Sections were rinsed, dehydrated, cleared in xylene, and mounted with synthetic resin.

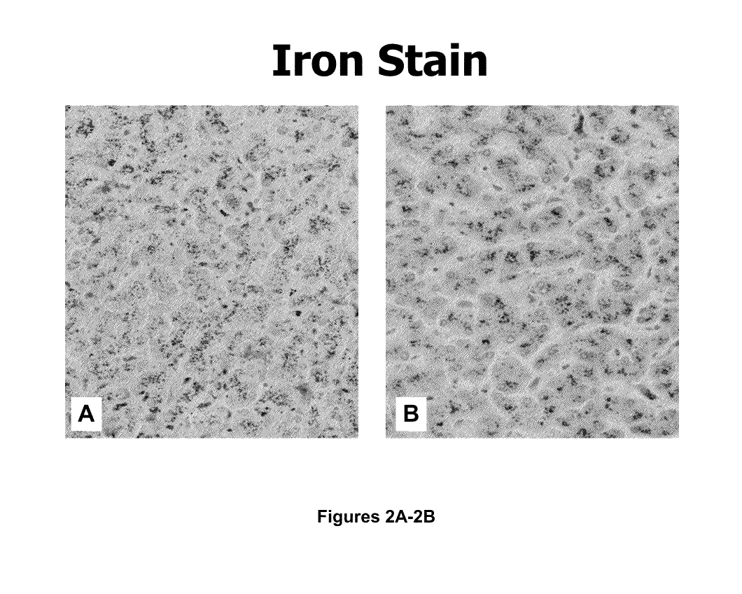

FIGS. 2A and 2B show a comparison of the ferric iron staining in liver tissue with nuclear fast red counterstain (FIG. 2A) and 0.1% pararosaniline nuclear counterstain (“strong fast red”) (FIG. 2B). The nuclear staining achieved with the 0.1% pararosaniline solution is brighter than that of the nuclear fast red and shows superior tissue architecture. A sharper and clearer contrast between the primary stain (ferric iron staining) and secondary stain (nuclear staining) is observed with the pararosaniline nuclear stain of Example 1 compared to the nuclear fast red stain of Comparative Example 2. In addition, no masking of the primary stain or non-specific staining was observed with the pararosaniline nuclear stain.

### Example 5

### Microwave Alcian Blue Staining for Acidic Mucins with Nuclear Counterstain in Small Bowel Tissue

Small bowel tissue was fixed in 10% buffered neutral formalin, embedded in paraffin, and sections were cut at 5 μm. Slides containing the liver tissue sections were deparaffinized and hydrated in distilled water. Sections were placed in 3% acetic acid for three minutes and then placed in 1.0% alcian blue solution (alcian blue in 3% acetic acid) and microwaved for 3 minutes (60 W). The slides were dipped several times and allowed to remain in the hot solution (65° C.) for 5 minutes. Following incubation in the alcian blue solution, the slides were washed in running tap water for 1 minute and rinsed in distilled water. Slides were counterstained with 0.1% nuclear fast red solution of Comparative Example 2 for 5 minutes or the 0.1% pararosaniline solution of Example 1 for 20 seconds. Sections were rinsed, dehydrated, cleared in xylene, and mounted with synthetic resin.

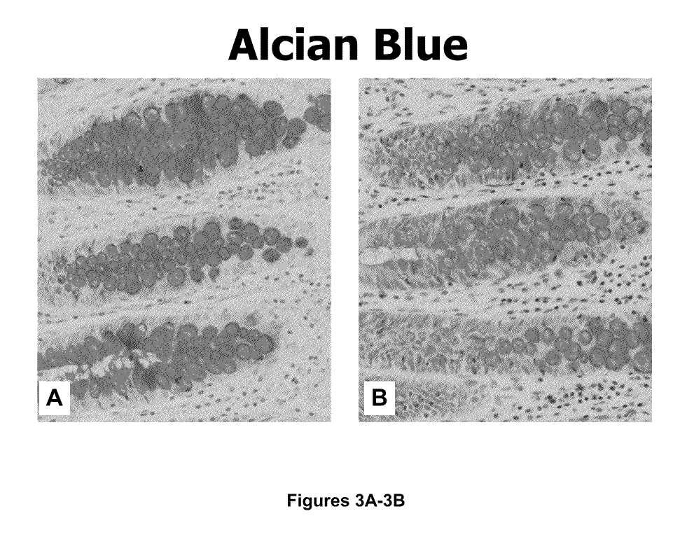

FIGS. 3A and 3B show a comparison of the alcian blue staining of acidic mucins in small bowel tissue with nuclear fast red counterstain (FIG. 3A) and 0.1% pararosaniline nuclear counterstain (“strong fast red”) (FIG. 3B). The nuclear staining achieved with the 0.1% pararosaniline solution is brighter and more punctuate than that achieved with the nuclear fast red. A sharper and clearer contrast between the primary stain (alcian blue staining) and secondary stain (nuclear staining) is observed with the pararosaniline nuclear stain of Example 1 compared to the nuclear fast red stain of Comparative Example 2. In addition, no masking of the primary stain or non-specific staining was observed with the pararosaniline nuclear stain.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A composition for detecting nucleic acids in a biological sample comprising:
a pH buffering reagent that maintains the composition at a pH of less than 5;
a solubilizing reagent;
a basic dye; and
an aqueous medium.

2. The composition of claim 1, wherein the pH buffering reagent is selected from the group consisting of lactic acid, acetic acid, citrate acid, oxalic acid, formic acid, hydrochloric acid, acetate buffer, citric acid/disodium phosphate buffer, sulfuric acid/sodium phosphate buffer, malonic acid/sodium hydroxide buffer, sodium acetate/acetic acid buffer, acid phosphate/hydrochloric acid buffer, and hydrochloric acid/sodium citrate buffer.

3. The composition of claim 1, wherein the pH buffering reagent comprises lactic acid at a concentration of between about 0.5% to about 0.7% by volume.

4. (canceled)

5. The composition of claim 1, wherein the pH buffering reagent comprises acetic acid at a concentration of between about 1% to about 5% by volume.

6. (canceled)

7. The composition of claim 1, wherein the solubilizing reagent comprises polysorbate 20 (TWEEN 20) or Triton X-100®.

8. The composition of claim 1, wherein the solubilizing reagent comprises polysorbate 20 or TritonX-100® at a concentration of between about 0.01% to about 0.04% by volume.

9. (canceled)

10. The composition of claim 1, wherein the basic dye is selected from the group consisting of pararosanilin, rosaniline, fuchsine, new fuchsine, magenta II, safranin O, neutral red, rhodamine B, pyronin B, pyronin G, iodonitrotetrazolium chloride, crystal violet, Victoria blue 4R, Victoria blue B, Victoria blue R, methylene blue, Nile blue A, toluidine blue O, azure A, azure B, azure C, nitro blue tetrazolium, night blue, alcian blue, gallamin blue, gallocyanin, thionin, Hoffman's violet, methyl violet 2B, methyl violet 6B, mauveine, ethyl violet, malachite green, methylene green, methyl green, iodine green, Thioflavine T, alcian yellow, Auramine O, and bismark brown Y.

11. (canceled)

12. The composition of claim 1, wherein the basic dye is present at a concentration of between 0.02% and 0.2%.

13. The composition of claim 1, wherein the basic dye comprises pararosanilin at a concentration of about 0.1% by weight.

14. (canceled)

15. The composition of claim 1, wherein the composition comprises a pH of between about 2 to about 3.

16.-17. (canceled)

18. The composition of claim 1, wherein the composition is suitable for staining nucleic acids in a biological tissue or cell sample.

19. A method of detecting nucleic acids in a biological sample comprising:
exposing the biological sample to a composition of claim 1 under conditions effective to label the nucleic acids in the biological sample; and
viewing the biological sample using light microscopy to detect the nucleic acids in the biological sample.

20. (canceled)

21. The method according to claim 19, wherein the biological sample is a histological plant or animal cell sample.

22.-24. (canceled)

25. The method according to claim 19 further comprising:
incubating the biological sample with one or more solutions capable of differentially labeling non-nucleic acid cellular components in the biological sample prior to said exposing and viewing the labeled nucleic acids and differentially labeled non-nucleic acid cellular components in the biological sample simultaneously.

26-27. (canceled)

28. A kit comprising:
a composition of claim 1 and instructions for using the composition for detecting nucleic acids in a biological sample.

29. The kit according to claim 28, further comprising:
one or more histological staining solutions for labeling non-nucleic acid cellular elements in the biological sample.

30. (canceled)

31. A composition for detecting nucleic acids in a biological sample consisting of:
about 0.5% to about 5% by volume of a pH buffering reagent that maintains the composition at a pH of less than 5;
about 0.008% to about 0.05% by volume of a solubilizing reagent;
about 0.02% to about 0.2% by weight of a basic dye; and
an aqueous medium.

32. (canceled)

33. The composition of claim 31, wherein the basic dye is selected from the group consisting of pararosanilin, rosaniline, fuchsine, new fuchsine, magenta II, safranin O, neutral red, rhodamine B, pyronin B, pyronin G, iodonitrotetrazolium chloride, crystal violet, Victoria blue 4R, Victoria blue B, Victoria blue R, methylene blue, Nile blue A, toluidine blue O, azure A, azure B, azure C, nitro blue tetrazolium, night blue, alcian blue, gallamin blue, gallocyanin, thionin, Hoffman's violet, methyl violet 2B, methyl violet 6B, mauveine, ethyl violet, malachite green, methylene green, methyl green, iodine green, Thioflavine T, alcian yellow, Auramine O, and bismark brown Y.

34. (canceled)

35. A method of detecting nucleic acids in a biological sample comprising:
exposing the biological sample to a composition of claim 31 under conditions effective to label the nucleic acids in the biological sample; and
viewing the biological sample using light microscopy to detect the nucleic acids in the biological sample.

36.-40. (canceled)

41. The method according to claim 35 further comprising:
incubating the biological sample with one or more solutions capable of differentially labeling non-nucleic acid cellular components in the biological sample prior to said exposing and
viewing the labeled nucleic acids and differentially labeled non-nucleic acid cellular components in the biological sample simultaneously.

42. (canceled)
